(19) United States
(12) Patent Application Publication
(10) Pub. No.: US 2007/0122445 A1
(43) Pub. Date: May 31, 2007

(54) Absorbable fatigue-enduring phosphate composites

(76) Inventor: Shalaby W. Shalaby, Anderson, SC (US)

Correspondence Address: LEIGH P. GREGORY;ATTORNEY AT LAW, PO BOX 168, CLEMSON, SC, 29633-0168, US

(21) Appl. No.: 11/598,501

(22) Filed: Nov. 13, 2006

Related U.S. Application Data
(60) Provisional application No. 60/740,006, filed on Nov. 28, 2005.

(51) Int. Cl. A61K 33/42; C04B 28/34; A61F 2/02

(52) U.S. Cl. 424/423; 106/691; 424/602; 977/906

(57) ABSTRACT

Precursors of fatigue-enduring phosphate composites, particularly useful as self-hardening bone cement, are formed of a calcium phosphate cement grafted with trimethylene carbonate, phosphate glass, a liquid amine-bearing derivative poly(ethylene glycol-b-propylene glycol) and a liquid C-succinylated polyalkylene glycol and preferably contain at least one bioactive agent.

### CROSS-REFERENCE TO RELATED APPLICATIONS

The present application claims the benefit of prior provisional application Ser. No. 60/740,006, filed Nov. 25, 2005.

### FIELD OF THE INVENTION

This present invention is directed to precursors of absorbable phosphate composites comprising necessary ingredients for use in many orthopedic applications and particularly as a self-hardening, absorbable phosphate bone cement that exhibits, in part, a number of the desirable physical properties of the current non-absorbable methyl methacrylate-based bone cement. Particularly, the present composites exhibit high tensile, torsional and flexural strength, impact resistance, and fatigue endurance, while having key absorption and osteoconductive properties. Additionally, the present inventive bone cement is expected to be (1) more biocompatible than its poly(methyl methacrylate) counterpart that often contains toxic levels of leachable methyl methacrylate; (2) more resistance to fracture than traditional phosphate bone cements (CPC); and (3) more capable of supporting bone regeneration than both the traditional poly(methyl methacrylate) and CPC. Additionally, the present inventive composites can be used as absorbable bone fillers or substitutes.

### BACKGROUND OF THE INVENTION

For some time, calcium phosphate cement (CPC), in which at least one dry component and a liquid are combined to form a flowable, paste-like material that is capable of setting into a solid calcium phosphate product, has held great promise for use as a structural material in orthopedic, cranio-maxillofacial, dental, and related fields. And, there has been a consistent need to have a flowable material that can be injected into a cancellous bone void, which then undergoes self-setting into a solid calcium phosphate mineral product that is capable of withstanding physiological loads. Materials that set into solid calcium phosphate mineral products have been of particular interest as such products can closely resemble the mineral phase of bone and are potentially remodelable, and hence, can be valuable in orthopedic and related fields. Relevant patents, which address different aspects of different phosphate-based compositions and their potential use in dental and orthopedic applications, include U.S. Pat. Nos. 5,037,639; 5,129,905; 5,178,845; 5,336,264; 5,496,399; 5,503,212; 5,508,342; 5,560,176; 5,569,442; 5,571,493; 5,676,976; 5,580,623; 5,683,461; 5,683,496; and 5,697,981.

Patents dealing with calcium phosphate cement comprising antimicrobial agents, which are particularly relevant to segments of the present invention, include U.S. Pat. No. 5,968,253. The latter teaches a flowable, paste-like composition capable of setting during clinically acceptable periods of time into an antimicrobial agent-loaded apatite product having sufficient compressive strength to serve as a cancellous bone structure. Compositions, subject of U.S. Pat. No. 5,968,253, are prepared by combining dry ingredients with a physiologically acceptable lubricant and antimicrobial agent where the dry ingredients comprise at least two different calcium phosphates and can be used for orthopedic, dental, and cranio-maxillofacial applications.

Due to its superior biocompatibility and osteoconductivity, calcium phosphate cement (CPC) has been used as implants or filling materials in dental and bone prostheses and the like. However, CPC suffers from two key clinically undesirable features. First, it has a long setting time, as for example when CPC is made by mixing TTCP (a reaction product of CaCO₃ and Ca₂P₂O₇ having the molecular formula: Ca₄(PO₄)₂O) and dicalcium phosphate anhydrous (monetite, DCPA) mixed powders with a dilute phosphate-containing solution to obtain hydroxyapatite (HA). The resulting implants were noted to have a compressive strength up to 51 MPa. However, in dental applications, the setting (or hardening) time was about 30 minutes and mechanical properties were inferior to enamel, tooth, and metal composites. Because of their long setting time, the implants can be flushed away by body fluid before hardening. This led to the pursuit of the art described in U.S. Pat. No. 6,379,453, which dealt with a process for producing fast-setting bioabsorbable calcium phosphate cement (CPC), and in particular, a process including a pre-heat treatment step to generate uniformly distributed submicron-sized apatite seeds.

Although CPC and associated systems were developed initially as more biocompatible substitutes to the more commonly used poly(methyl methacrylate) bone cement, contemporary investigators, including the present inventor, viewed phosphate-based materials as a viable source of biomaterials which can be used not only as bone cement but also as bone fillers or substitutes. Extension of the phosphate-based materials technology entailed development of (1) absorbable (or resorbable) phosphate glasses; (2) calcium phosphate systems with modulated absorption profiles; (3) highly osteoconductive ceramics for supporting bone regeneration; and (4) hybrid inorganic and organic systems as implants that can absorb and allow their replacement by natural bone. Most relevant examples of these developments, which are also relevant to the subject of the present invention, are those disclosed in the following patents.

U.S. Pat. No. 5,874,509 in which the present inventor and his coworkers described an inorganic glass having an aliphatic polymer covalently bonded to the surface thereof for providing improved adhesion to a matrix polymer when the glass is employed as a filler in composites, as well as for the formation of a composite absent a separate matrix polymer. The polymer is grafted onto the glass by a method which includes the steps of pretreating the surface of the glass with an activator which introduces an activating moiety to the surface, functionalizing the pretreated surface by bonding functional moieties thereto, and polymerizing a cycloaliphatic monomer onto the functionalized surface in the presence of a ring opening polymerization catalyst.

U.S. Pat. No. 6,287,341 and its teaching was a response to a recognized need (a) for a synthetic bone material that more closely mimics the properties of naturally occurring minerals in bone, and (b) to provide synthetic bioceramics which are completely bioresorbable and biocompatible. The use of a resorbable calcium phosphate in biomedical devices was expected to provide many advantages over alternative conventional materials. For instance, it eliminates the need for post-therapy surgery to remove the device and degrades in the human body to biocompatible, bioresorbable products. In effect, U.S. Pat. No. 6,287,341 dealt with a method for treating a bone defect by identifying a bone site suitable for receiving; and introducing a strongly resorbable, poorly crystalline apatitic calcium phosphate at the implant site, whereby bone is formed at the site. Thus, a bone defect may be treated by identifying a bone site suitable for receiving an implant and introducing a hydrated precursor of a strongly resorbable, poorly crystalline apatitic calcium phosphate at the implant site, whereby the hydrated precursor is converted in vivo to a poorly crystalline apatitic calcium phosphate and whereby bone is formed at the implant site. The implant site can be a tooth socket, non-union bone, bone prosthesis, osteoporotic bone, intervertebral space, alveolar ridge, or bone fracture.

U.S. Pat. No. 6,703,038 is directed to an injectable bone substitute material containing non-ceramic hydroxyapatite cement. More specifically, the '038 patent deals with a bone substitute material which comprises a soft matrix, living cells, and a setting matrix comprising non-ceramic hydroxyapatite cement. The bone substitute material can be injected minimally invasively into a bone defect with a suitable injection apparatus. In an embodiment of this patent, living cells are mixed with a fibrinogen solution and then with a thrombin solution to form the soft matrix, and the soft matrix is mixed with an aqueous solution of non-ceramic hydroxyapatite cement to obtain the bone substitute material which remains unsolidified until after application to a biological site.

Developments of the novel phosphate-based biomaterials described above as per the cited patent literature were paralleled by a number of relevant publications, outlined below.

A. D. Speirs et al. (Biomaterials, 26, 7310, 2005) used calcium phosphate cement composites in revision hip arthroplasty. The authors reported that the addition of a bioresorbable cement to an allograft layer may improve the properties of the layer, preventing early subsidence seen in some clinical studies of impaction allografting, and therefore improving the clinical results.

P. Q. Ruhe and coworkers (J. Biomed. Mater. Res., 74-A, 533, 2005) prepared poly(dl-lactic-co-glycolic acid)/calcium phosphate cement (PLGA/CPC) composites and evaluated their biocompatibility and degradation. Results of the study led to the conclusion that PLGA microparticles embedded in PLGA/Ca—P cement composites form macropores within the Ca—P cement framework after PLGA degradation in vivo. An average pore size of 73±27 μm is sufficient to allow ingrowth of the soft and hard tissues. The PLGA/Ca—P cement composites show excellent osteocompatibility in weight ratios of 15/85 and 30/70 PLGA/Ca—P cement. Histological aspects suggest that 30/70 PLGA/Ca—P cement composites show the most favorable biologic response in this model.

P. Q. Ruhe and coworkers (J. Controlled Release, 106, 162, 2005) loaded the composites of PLGA/CPC as microparticles with rhBMP-2 and investigated their in vivo release profile in rats. Results of their study, using ¹³¹I-labeled rhBMP-2 led to the conclusion that PLGA/Ca—P cement composites are sustained, slow release vehicles for rhBMP-2 delivery in vivo. Release profiles of rhBMP-2 loaded PLGA/Ca—P cement composites can be influenced to a limited extent by variation of molecular weight of the PLGA used for microparticle preparation and administration method of the rhBMP-2. Most likely, release of rhBMP-2 from the composite is slowed down by an avid interaction of rhBMP-2 and Ca—P cement after rhBMP-2 release from PLGA microparticles.

H. H. K. Xu and coworkers (Biomaterials, 25, 1029, 2004) investigated the synergistic reinforcement of in situ hardening composite calcium phosphate composite scaffold for bone tissue engineering. The study was directed toward increasing the strength and toughness of CPC while creating micropores suitable for cell infiltration and bone ingrowth and to investigate the effects of chitosan (applied as a solution) and a mesh (made of a 90/10 glycolide/l-lactide copolymer) reinforcement on the composite properties. Results of this study led the authors to make the following statements:

(1) Substantial synergistic effects via combining reinforcement agents were quantitatively demonstrated for biomaterials for the first time. The reinforcement efficacy from mesh and chitosan together was not only much more than the reinforcement from either mesh or chitosan alone. It was also much more than the reinforcement gained from mesh plus that from chitosan. Such microstructural design methods may have wide applicability to the improvement of other biomaterials.

(2) The incorporation of absorbable meshes into CPC had a two-fold benefit: (a) providing the needed early strength and toughness to the implant while tissue regeneration was occurring; and (b) creating macropores for bone ingrowth after mesh dissolution. The highly interconnected long cylindrical pores from mesh dissolution may be advantages for cell infiltration and active bone ingrowth than the conventional random pores and spherical pores.

(3) Macropores tended to precipitously degrade the strength of the implants. However, the novel macroporous CPC was stronger than the conventional CPC without macropores. The CPC scaffold had a strength within the range for sintered porous hydroxyapatite implants and higher than the strength for cancellous bone. Compared to sintered hydroxyapatite, the advantages of the CPC scaffold included its moldability, ability to be injected and self-harden in situ in the bone cavity, ability to conform to complex and irregular cavity shapes without machining, and bioresorbability.

To improve compatibility of HA microparticles with a polylactide matrix in their respective composites [Z. Hong et al., Polymer, 45, 6699 (2005)] investigators grafted l-lactide directly onto the surface of HA microparticles prior to mixing the polylactide matrix.

Growing needs for broader application of biocompatible phosphate-based materials in general, and the phosphate cement in particular as useful biomaterials for orthopedic, dental, and cranio-maxillofacial applications and acknowledgement of failure of the prior art, in spite of the above cited patents and technical literature, to conceive a novel approach to meet these needs provided a strong incentive to explore the subject of the present invention. Accordingly, this invention deals with absorbable phosphate composites comprising necessary ingredients for use in many orthopedic applications and particularly as an absorbable phosphate bone cement that exhibits, in part, a number of the desirable physical properties of the non-absorbable methyl methacrylate-based bone cement such as high tensile, torsional, and flexural strength, impact resistance, and fatigue endurance, while having key absorption and osteoconductive properties. Additionally, the bone cement subject of this formulation is expected to be (1) more biocompatible than its poly(methyl methacrylate) counterpart that often contains toxic levels of leachable methyl methacrylate; (2) more resistance to fracture than traditional phosphate bone cements; and (3) more capable of supporting bone regeneration than both the traditional poly(methyl methacrylate) and CPC. Additionally, composites subject of this invention can be used as bone fillers or substitutes.

### SUMMARY OF THE INVENTION

Generally, the present invention is directed to absorbable precursors of fatigue-enduring phosphate composites for use as bone cement for orthopedic, dental, and cranio-maxillofacial applications as well as for bone fillers or substitutes. In an important aspect, the present invention is directed to absorbable precursors of fatigue-enduring phosphate cement composites which are formed of calcium phosphate cement micro-/nanoparticulates surface grafted with trimethylene carbonate and up to about 20 mole percent of at least one cyclic lactone such as glycolide, lactide, ε-caprolactone or a morpholinedione, phosphate glass micro-/nanoparticulates, a liquid amine derivative of poly(ethylene glycol-b-propylene glycol), and a liquid C-succinylated polyalkylene glycol such as polyethylene glycol and its block copolymers with polypropylene glycol, wherein the phosphate-containing components represent at least 50 weight percent of the total mass.

Preferably, the phosphate glass micro-/nanoparticulates are derived from a calcinized mixture comprising CaO and P₂O₅ at 1-30, 1-70 weight percent, respectively, and at least two of the oxides from the group represented by Na₂O, K₂O, ZnO, and SiO₂ at 1-50, 1-25, 1-40, and 1-30 weight percent, respectively. It is also preferred that the phosphate glass micro-/nanoparticulates contain at least one bioactive agent. The bioactive agent can be one or more antimicrobial agents selected from the groups represented by gentamicin, tobramycin, vancomycin, clindamycin, and other functionally similar agents. The bioactive agent may also be one or more bone growth promoters selected from the group represented by a bone morphogenic protein such as rh-BMP-2, fibroblast derived growth factor (FGF), an osteoblast activating oligopeptide, and other functionally similar agents.

Preferably, the phosphate glass micro-/nanoparticulates have a water solubility of 20-100 percent, more preferably at least about 50 percent, (weight/volume) in water at 25° C. Thus, such compositions form an aqueous, flowable, paste-like composition capable of self-hardening as bone cement following injection at a subject biological site. Such compositions can contain bioactive agents of the antimicrobial type or those which are capable of accelerating tissue regeneration.

The present absorbable precursors are capable of forming fatigue-enduring, phosphate-containing composites which may serve as bone fillers or substitutes. A clinically important aspect of this invention deals with using the absorbable precursors of fatigue-enduring phosphate composites described herein in applications such as those dealing with tooth sockets, non-union bones, bone prostheses, osteoporotic bones, intervertebral space, alveolar ridges, bone fractures, and artificial joint replacements.

### DETAILED DESCRIPTION OF PREFERRED EMBODIMENTS

The present invention is directed to precursors of absorbable phosphate composites and in part, their use as self-hardening, absorbable bone cement with preferred physical and biophysical properties for clinical use in orthopedic and dental applications. As compared to traditional non-absorbable polymeric methyl methacrylate bone cement (PMMA-BC) and currently available calcium phosphate cement bone cement (CPC-BC), the present invention provides bone cement that absorbs during pre-determined periods of time to allow gradual load transfer, osteoconduction, and eventual bone regrowth at the implant site. Further, the present inventive precursors yield self-hardening cements that solidify in an aqueous environment with minimum heat generation to avoid the tissue necrosis often encountered in the curing of the PMMA-BC precursor and which contain no leachable toxic methyl methacrylate as in the case of PMMA-BC. Further, the present inventive precursors provide self-hardening bone cements with organic fillers representing more than 50 weight percent and hence conform to irregular site geometry upon solidifying and integrating tightly with the adjacent tissue with practically no shrinkage at the application site as compared with the expected shrinkage due to the monomer conversion to a polymer as in the case of PMMA-BC. Also, the present inventive precursor provide bone cements that exhibit exceptional degrees of toughness, measured in terms of work to break, higher flexural, and torsional strength as compared to the totally inorganic CPC-BC which suffers from poor impact, inferior flexural and torsional strength, and totally unacceptable fragility for use in high or even moderate-to-low load-bearing applications as in the case of many orthopedic implants. Additionally, the present inventive precursors contain solid micro-/nanoparticulate phosphate fillers exhibiting at least a bimodal particle size distribution to maximize their packing index leading to high density, strong cured material and present to the biological site bone cement that forms under mild conditions and is made of both hydrophilic polar and lipophilic, less polar components, which are a liberal and facile incorporation of different types of bioactive agents, such as the antimicrobial types and those capable of promoting bone regeneration and growth.

Technologically, elements of this invention bring to bear a number of novel concepts which include: (1) grafting low melting solid- or liquid-forming organic polymer chains which can bind chemically to inherently soft and compliant components to high modulus and fragile solid microparticulates, thus allowing the modulation of their physicomechanical properties so as to absorb prevailing mechanical stresses and improve the impact, flexural and torsional strength of load-bearing implants thereof; (2) using combinations of organic polycationic (e.g., amine-bearing polyethers) and polyanionic (e.g., carboxyl-bearing and specifically C-succinylated polyethers) as components of the precursors to induce more controlled system gelation and self-hardening of the cement as compared with those based on totally inorganic ions, as in the case of CPC-BC and similar systems; (3) the ability for ionic binding of basic or acidic bioactive agents on the carboxyl-bearing and amine-bearing polyether chains and hence, the ability of modulating their release into the surrounding tissue at the implant site; (4) using absorbable glasses which provide a source of phosphate and calcium ions for promoting bone mineralization; (5) using absorbable calcium zincophosphate absorbable glasses to release zinc ions which are known to promote bone growth; (6) using absorbable calcium silicophosphates which are capable of making available limited amounts of silicate moieties which can contribute to the strengthening of newly formed bone; and (7) using bimodal or multimodal size distribution of the filler micro-/nanoparticles to maximize the packing factor or density of the cement upon formation and regulate more precisely its bioabsorption profile.

The invention is directed to absorbable precursors of fatigue-enduring phosphate composites for use as bone cement for orthopedic, dental, and cranio-maxillofacial applications as well as for bone fillers or substitutes. Generally, the present absorbable precursors of fatigue-enduring phosphate cement composites contain calcium phosphate cement micro-/nanoparticulates which are surface grafted with trimethylene carbonate and up to about 20 mole percent of at least one cyclic lactone such as glycolide, lactide, ε-caprolactone and morpholinedione, phosphate glass micro-/nanoparticulates, a liquid amine derivative of poly(propylene glycol-b-ethylene glycol-b-propylene glycol), and a liquid C-succinylated polyalkylene glycol such as polyethylene glycol and its block copolymers with polypropylene glycol, wherein the phosphate-containing components represent at least 50 weight percent of the total mass. The C-succinylation of the polyalkylene glycol is achieved through free-radically induced maleation of the polymeric chain and subsequent hydrolysis of the maleic anhydride side groups to yield succinic acid side groups.

Preferably, the phosphate glass micro-/nanoparticulates are derived from a calcinized mixture comprising CaO and P₂O₅ at 1-30, 1-70 weight percent, respectively, and at least two of the oxides from the group represented by Na₂O, K₂O, ZnO, and SiO₂ at 1-50, 1-25, 1-40, and 1-30 weight percent, respectively. It is also preferred that the phosphate glass micro-/nanoparticulates contain at least one bioactive agent. The bioactive agent can be one or more antimicrobial agents selected from the groups represented by gentamicin, tobramycin, vancomycin, clindamycin, and other functionally similar agents. The bioactive agent may also be one or more bone growth promoters selected from the group represented by a bone morphogenic protein such as rh-BMP-2, fibroblast derived growth factor (FGF), an osteoblast activating oligopeptide, and other functionally similar agents.

Preferably, the phosphate glass micro-/nanoparticulates have a water solubility of 20-100 percent, more preferably at least about 50 percent, (weight/volume) in water at 25° C. Thus, such compositions form an aqueous, flowable, paste-like composition capable of self-hardening as bone cement following injection at a subject biological site. Such compositions can contain bioactive agents of the antimicrobial type or those which are capable of accelerating tissue regeneration.

The present absorbable precursors are capable of forming fatigue-enduring, phosphate-containing composites which may serve as bone fillers or substitutes. A clinically important aspect of this invention deals with using the absorbable precursors of fatigue-enduring phosphate composites described herein in applications such as those dealing with tooth sockets, non-union bones, bone prostheses, osteoporotic bones, intervertebral space, alveolar ridges, bone fractures, and artificial joint replacements.

Further illustrations of the present invention are provided by the following examples:

### EXAMPLE 1

### General Method of Preparing Water-Soluble Calcium Phosphate and Absorbable Calcium Silicophosphate and Zincophosphate Glasses and Their Size Reduction

Preparation of these glasses requires the use of certain intermediate compounds which, upon heating in the early stages of glass formation produce their respective oxides, water vapor, carbon dioxide, and/or ammonia gas. This requires adjusting the initial stoichiometry of the glass precursors to account for the expected initial mass loss due to vapor or gas evolution and staging the heating process to prevent premature, uncontrolled gas evolution at the early stages of glass formation. An illustration of the changes in mass of typical gas- or vapor-producing starting compounds upon thermal conversion to their respective oxides is given below:

| Starting Compound | Vapor or Gas Evolved | Resulting Oxide |
|---|---|---|
| K₂H₂PO₄ | H₂O | P₂O₅ |
| Na₂H₂PO₄ | H₂O | Na₂O, P₂O₅ |
| (NH₄)H₂PO₄ | H₂O, NH₃ | P₂O₅ |
| SiO₂•xH₂O | H₂O (10.6 wt %) | SiO₂ |

To form these glasses, predetermined weights of the powdered starting components are thoroughly mixed and transferred into porcelain crucibles (Coors, 15 mL capacity) and heated in a Branstead Thermolyne-62700 muffle furnace from room temperature to 300° C. at a rate of approximately 10° C./min., followed by a heating rate of 15°/min. to reach 500° C. During this heating period, the mixture undergoes loss of water, carbon dioxide, and/or ammonia, depending on its composition. Melting then occurs between 700° C. and 1100° C. Once the melt appears clear and homogeneous (usually between 800-900° C.), the glass is poured onto a steel mold and annealed at 200° C. for 15-30 minutes and allowed to slowly cool to room temperature. Melts are preferably poured onto a stainless steel plate at the lowest temperature possible to reduce volatilization of P₂O₅.

For size reduction, the resulting glass is first ground at room temperature using a Wiley Mill and sieved to isolate two crops of particles having average diameters not exceeding 100μ and 500μ. The two crops are then subjected separately to cryogenic size reduction at liquid nitrogen temperature using a Spex 6850 Freezer Mill. In both cases, the size reduction is pursued for the required period of time to produce five new crops having different particle size distribution, corresponding namely to diameter ranges of about <1 to 5μ average, 5 to 10μ, 10 to 50μ, 50 to 100μ, and 100 to 300μ for the first, second, third, fourth, and fifth crops, respectively. The final products are isolated and dried at 60° C. under reduced pressure, prior to charactering and mixing as components of the precursors system.

### EXAMPLE 2

### Characterization of Phosphate Glasses

Glasses produced according to Example 1 are characterized for (1) identity and composition using FTIR, elemental microanalysis, and electron spectroscopy for chemical analysis (ESCA); (2) thermal property and morphology using high temperature DSC and X-ray diffraction methods; (3) particle size and particle size distribution and surface morphology using particle size analyzer and scanning electron microscopy; and (4) water solubility in terms of water loss in deionized water at 25° C.

### EXAMPLE 3

### Preparation of a Water-Soluble Calcium Phosphate Glass Composition

Using the general method for glass formation and size reduction described in Example 1, a water-soluble calcium phosphate having the following molar composition of the oxide precursors is prepared: P₂O₅, 62%; Na₂O, 15%; CaO, 18%; ZnO, 5%

### EXAMPLE 4

### Preparation of an Absorbable Calcium Silicophosphate Glass Composition

Using the general method for glass formation and size reduction described in Example 1, a calcium silicophosphate having a molar composition of the oxide precursors as given below, is prepared: P₂O₅, 62%; Na₂O, 8%; K₂O, 8%; CaO, 18%; SiO, 4%

### EXAMPLE 5

### Preparation of an Absorbable Calcium Zincophosphate Glass Composition

Using the general method for glass formation and size reduction described in Example 1, a calcium zincophosphate having a molar composition of the oxide precursors as given below, is prepared: P₂O₅, 62%; Na₂O, 6%; K₂O, 6%; CaO, 18%; ZnO, 8%

### EXAMPLE 6

### Preparation and Size Reduction of a Typical Calcium Phosphate Cement

A 1:1.27 mixture (weight ratio) of calcium pyrophosphate (Ca₂P₂O₇) powder and calcium carbonate (CaCO₃) powder are stirred in ethanol for 24 hours at 25° C. to insure uniform dispersion. The ethanol dispersion is heated in a rotary evaporator under reduced pressure at temperatures ranging between 25° C. to 100° C. until no ethanol could be collected. The powder mixture is transferred into a ceramic crucible and heated to react inside a muffle furnace according to the following scheme of temperature range/heating rate: 25-500° C./15 min.; 500-800° C./20 min.; 800-1200° C./10 min.; 1200-1400° C./4 min. The heating is maintained at 1400° C. for 12 hours. The furnace temperature is reduced to 1000° C., heating is discontinued, and then turned off and the product is allowed to cool to room temperature. The resulting powder (TTCP) having the molecular formula Ca₄(PO₄)₂O is sieved in a ball mill for 12 hours and blended with dried dicalcium phosphate (or dicalcium phosphate anhydrous, DCPA) (CaHPO₄) in 1:1 molar ratio to obtain the desired CPC powder. The average particle size of the CPC is determined and further reduced cryogenically, sieved, and dried as described for the absorbable phosphate glasses in Example 1. The dry CPC is characterized as discussed in Example 2. The fraction of CPC particles having an average particle size distribution of <1 to 5μ or simply the nano-/microparticles is handled using specially designed equipment.

### EXAMPLE 7

### Surface Grafting Nano-/Microparticulate CPC with Trimethylene Carbonate: Preparation of PTMC-g-CPC

A flame dried stainless steel reactor equipped for mechanical agitation is charged, under a dry nitrogen atmosphere, with predried (at 130° C. under reduced pressure for 16 hours) CPC nano-/microparticles (30.3 g) and a solution of trimethylene carbonate (TMC) (30.3 g) in anhydrous decahydronaphthalene (300 mL) containing stannous octanoate (4.01 mg, 1 mmole). The contents of the reactor are heated while stirring under dry nitrogen atmosphere to reach 160° C. and maintained at that temperature until practically all the monomer is consumed (as determined by gel-permeation chromatography). At the conclusion of the reaction period, the reaction mixture is cooled to room temperature and polytrimethylene carbonate grafted CPC particles (PTMC-g-CPC) are separated by centrifugation at 20,000 rpm and rinsed several times with methylene chloride to remove traces of homopolymeric TMC. This is determined by GPC analysis of the methylene chloride rinses. The purified PTMC-g-CPC sediment is isolated and dried under reduced pressure at 25° C. until a constant weight is realized. The composition of the product is verified using elemental analysis and FTIR.

### EXAMPLE 8

### Surface Grafting of Nano-/Microparticulate CPC with an 80/20 Mixture of TMC/L-lactide (LL): Preparation of P-TMC/LL-g-CPC

The preparation of surface grafted CPC is conducted and the product is purified and isolated and characterized as described in Example 7 for PTMC-g-CPC with the exception of substituting the TMC by an 80/20 (molar) mixture of TMC/LL.

### EXAMPLE 9

### Surface Grafting of Nano-/Microparticulate CPC with a 90/10 Mixture of TMC/Glycolide(G): Preparation of P-TMC/G-g-CPC

The preparation of P-TMC/G-g-CPC is pursued and the product is purified, isolated, and characterized as described in Example 7 for PTMC-g-CPC with the exception of substituting the TMC by a 90/10 (molar) mixture of TMC/G and reducing the amount of stannous octanoate to 2.01 mg (0.5 mmole).

### EXAMPLE 10

### Preparation of C-Succinylated Polyethylene Glycol-600 (PEG-600-S): A Typical Method

The PEG-S is prepared by heating at 80° C. under dry nitrogen atmosphere a solution of dried PEG-20K (200 g, 0.01 mole) containing benzoyl peroxide (13.3 g, 0.055 mole) in dry dioxane (600 mL) maleic anhydride (9.8 g, 10.1 mole). The heating is continued until practically all the maleic anhydride is consumed (as determined by FTIR). At the conclusion of the reaction, the dioxane is removed by distillation under reduced pressure. To obtain the C-succinylated product, the maleated reaction product is treated with water (50 mL) and 50° C. until all the anhydride groups are converted to succinic acid side groups (as determined by FTIR). After removing the water in the C-succinylated product, the dry mass was triturated with cold 2-propanol to remove races of unreacted maleic anhydride.

### EXAMPLE 11

### Preparation of a Typical Fatigue-enduring Phosphate Composite: A General Procedure

This entails preparation of a solid mixture “A” comprising pre-sized powders in the desirable amounts and particle size distributions as outlined below. Additionally, a soluble phosphate solution “B” is prepared using deionized water and the precursors noted below. Components of mixture “A” are blended thoroughly to insure composition uniformity. An aliquot of mixture “A” is mixed mechanically with a predetermined volume of aqueous solution “B” (precooled to a temperature of about 10 to 20° C.) for 1 to 5 minutes at room temperature and atmospheric pressure to produce a flowable paste. While mixing, Jeffamine-900, a poly(propylene glycol-b-ethylene glycol-b-propylene glycol) bis(2-aminopropyl ether) having a molecular weight of 900 Da, and then PEG-600-S (from Example 10) are added to the paste and the mixing continued for an additional 2 to 5 minutes at atmospheric pressure and then under reduced pressure until a degassed, flowable paste was formed. The resulting paste is then transferred to a small ram extruder equipped with a static mixer. The paste is then extruded into a test specimens mold and allowed to cure at 37° C. The cure time is determined. After reaching room temperature, the test specimens are removed from the mold and tested for their physicomechanical properties at 25° C. and 37° C.

Components of Solid Mixture “A”

| Component | Source | Component | Source |
|---|---|---|---|
| CPC | Example 3 | Calcium silicophosphate | Example 4 |
| PTMC-g-CPC | Example 7 | Calcium zincophosphate | Example 5 |

Components of Solution “B”

| Component | Source | Component | Source |
|---|---|---|---|
| Deionized Water | PMI | Water-soluble phosphate glass | Example 1 |

Preferred embodiments of the invention have been described using specific terms and devices. The words and terms used are for illustrative purposes only. The words and terms are words and terms of description, rather than of limitation. It is to be understood that changes and variations may be made by those of ordinary skill art without departing from the spirit or scope of the invention, which is set forth in the following claims. In addition it should be understood that aspects of the various embodiments may be interchanged in whole or in part. Therefore, the spirit and scope of the appended claims should not be limited to descriptions and examples herein.

What is claimed:

1. An absorbable precursor of fatigue-enduring phosphate composites, comprising:
calcium phosphate cement micro-/nanoparticulates surface grafted with trimethylene carbonate and up to about 20 mole percent of at least one cyclic lactone selected from the group consisting of glycolide, lactide, ε-caprolactone and a morpholinedione;
phosphate glass micro-/nanoparticulates;
a liquid amine derivative of poly(propylene glycol-b-ethylene glycol-b-propylene glycol); and
a liquid C-succinylated polyalkylene glycol, the polyalkylene glycol selected from the group consisting of polyethylene glycol and block copolymers of polyethylene glycol and polypropylene glycol.

2. An absorbable precursor of fatigue-enduring phosphate composites as set forth in claim 1 wherein the phosphate-containing micro-/nanoparticulate components comprise at least 50 weight percent of the total mass.

3. An absorbable precursor of fatigue-enduring phosphate composites as set forth in claim 1 wherein the phosphate glass micro-/nanoparticulates are derived from a calcinized mixture comprising from about 1 to about 30 weight percent of CaO, from about 1 to about 70 weight percent of P₂O₅, and at least two oxides selected from the group consisting of Na₂O, K₂O, ZnO, and SiO₂.

4. An absorbable precursor of fatigue-enduring phosphate composites as set forth in claim 3 wherein the phosphate glass micro-/nanoparticulates contain at least one bioactive agent.

5. An absorbable precursor of fatigue-enduring phosphate composites as set forth in claim 4 wherein the bioactive agent is selected from the group consisting of gentamicin, tobramycin, vancomycin, clindamycin, and combinations thereof.

6. An absorbable precursor of fatigue-enduring phosphate composites as set forth in claim 4 wherein the bioactive agent comprises at least one bone growth promoter selected from the group consisting of a bone morphogenic protein, fibroblast derived growth factor (FGF), an osteoblast activating oligopeptide, and combinations thereof.

7. An absorbable precursor of fatigue-enduring phosphate composites as set forth in claim 6 wherein the bone morphogenic protein comprises rh-BMP-2.

8. An absorbable precursor of fatigue-enduring phosphate composites as set forth in claim 3 wherein the phosphate glass microparticulates have a water solubility of from about 20 to about 100 percent in water at 25° C.

9. An absorbable precursor of fatigue-enduring phosphate composites as set forth in claim 8 wherein the phosphate glass microparticulates have a water solubility of at least about 50 percent in water at 25° C.

10. An absorbable precursor of fatigue-enduring phosphate composites as set forth in claim 9 as an aqueous, flowable, paste-like composition capable of self-hardening as bone cement following injection at a subject biological site.

11. An absorbable precursor of fatigue-enduring phosphate composites as set forth in claim 10 further comprising at least one bioactive agent.

12. An absorbable precursor of fatigue-enduring phosphate composites as set forth in claim 11 wherein the at least one bioactive agent comprises an antimicrobial agent.

13. An absorbable precursor of fatigue-enduring phosphate composites as set forth in claim 11 wherein the at least one bioactive agent is capable of accelerating tissue regeneration.

14. An absorbable precursor of fatigue-enduring, phosphate composites as set forth in claim 1 for use as bone fillers or substitutes.

15. An absorbable precursor of fatigue-enduring phosphate composite as set forth in claim 1 for use in clinical applications involving a member of the group consisting of tooth sockets, non-union bones, bone prostheses, osteoporotic bones, intervertebral space, alveolar ridges, bone fractures, and artificial joint replacements.
